(19) United States
(12) Patent Application Publication
(10) Pub. No.: US 2026/0338800 A1
(43) Pub. Date: Oct. 29, 2026

(54) METHOD FOR OBTAINING INFORMATION ON RISK OF DECLINE IN RENAL FUNCTION IN PATIENT WITH CHRONIC KIDNEY DISEASE, AND METHOD FOR ASSISTING DETERMINATION OF RISK OF DECLINE IN RENAL FUNCTION IN PATIENT WITH CHRONIC KIDNEY DISEASE

(71) Applicant: SYSMEX CORPORATION, Hyogo (JP)

(72) Inventors: Takehiro Hasegawa, Tokyo (JP); Yuko Saruta, Hamburg (DE)

(73) Assignee: SYSMEX CORPORATION, Hyogo (JP)

(21) Appl. No.: 19/660,102

(22) Filed: Apr. 27, 2026

(30) Foreign Application Priority Data
Apr. 28, 2025 (JP) 2025-074803

(51) Int. Cl. G01N 33/68; G16H 50/30

(52) U.S. Cl. CPC G01N 33/6893; G16H 50/30; G01N 2333/525; G01N 2333/54; G01N 2800/347; G01N 2800/52

(57) ABSTRACT

Provided is a method for obtaining information on a risk of a decline in renal function in a patient with chronic kidney disease, comprising measuring a biomarker in a blood sample obtained from the patient with chronic kidney disease, wherein the biomarker comprises at least one selected from the group consisting of IL-22 and TNF-α, and a measurement result of the biomarker serves as an index for the risk of the decline in renal function.

### CROSS REFERENCE TO RELATED APPLICATIONS

This application claims priority from prior Japanese Patent Application No. 2025-074803, filed on Apr. 28, 2025, entitled “METHOD FOR OBTAINING INFORMATION ON A RISK OF A DECLINE IN RENAL FUNCTION IN A PATIENT WITH CHRONIC KIDNEY DISEASE, METHOD FOR ASSISTING DETERMINATION OF A RISK OF A DECLINE IN RENAL FUNCTION IN A PATIENT WITH CHRONIC KIDNEY DISEASE, AND REAGENT KIT”, the entire content of which is incorporated herein by reference.

### TECHNICAL FIELD

The present invention relates to a method for obtaining information on a risk of a decline in renal function in a chronic kidney disease (CKD) patient. The present invention relates to a method for assisting determination of a risk of a decline in renal function in a patient with CKD.

The present invention relates to a reagent kit for use in these methods.

### BACKGROUND

CKD is a disease in which a state where presence of renal impairment is evident and/or a state where a glomerular filtration rate (eGFR or GFR) is less than 60 mL/min/1.73 m² continues for 3 months or more. In early CKD, there are few symptoms. However, even during the asymptomatic period, the pathological condition progresses and renal function declines. CKD patients eventually develop renal failure and may require dialysis treatment. CKD is diagnosed by GFR and albuminuria. KDIGO 2024 Clinical Practice Guideline for the Evaluation and Management of Chronic Kidney Disease, Kidney International (2024) Supplement, vol. 105, S117-S314 describes a classification table of severity of CKD based on GFR and albuminuria. Patients are classified based on this table, and a treatment method is determined according to the risk of death or end-stage renal failure. However, the classification according to the above classification table did not have sufficiently satisfactory performance for determining the risk of deterioration of renal function. Therefore, KDIGO 2024 Clinical Practice Guideline for the Evaluation and Management of Chronic Kidney Disease, Kidney International (2024) Supplement, vol. 105, S117-S314 also recommends evaluating the risk of renal failure in CKD patients using an externally validated risk equation using various variables such as age, sex, eGFR, and albuminuria.

### SUMMARY

An object of the present invention is to provide a means that makes it possible to determine a risk of a decline in renal function in a CKD patient based on a measurement result of a biomarker in a blood sample.

The present invention relates to a method for obtaining information on a risk of a decline in renal function in a patient with chronic kidney disease, comprising:
measuring a biomarker in a blood sample obtained from the patient with chronic kidney disease, wherein the biomarker comprises at least one selected from the group consisting of IL-22 and TNF-α, and a measurement result of the biomarker serves as an index for the risk of the decline in renal function.

The present invention relates to a method for assisting determination of a risk of a decline in renal function in a patient with chronic kidney disease, comprising:
measuring a biomarker in a blood sample obtained from the patient; and
determining the risk of the decline in renal function in the patient based on a measurement result, wherein the biomarker comprises at least one selected from the group consisting of IL-22 and TNF-α.

The present invention relates to a reagent kit for use in the above method, the reagent kit comprising a substance capable of specifically binding to IL-22 and/or a substance capable of specifically binding to TNF-α.

According to the present invention, a risk of a decline in renal function in a CKD patient can be determined.

### BRIEF DESCRIPTION OF THE DRAWINGS

FIG. 1A is a diagram showing an example of an appearance of a reagent kit.

FIG. 1B is a diagram showing an example of an appearance of a reagent kit.

FIG. 2A is an ROC (Receiver Operating Characteristic) curve showing determination accuracy when a risk of a decline in eGFR is determined based on an IL-22 concentration.

FIG. 2B is an ROC curve showing determination accuracy when a risk of a decline in eGFR is determined based on a TNF-α concentration.

FIG. 2C is an ROC curve showing determination accuracy when a risk of a decline in eGFR is determined based on a combination of a TNF-α concentration and an IL-22 concentration.

FIG. 3 is a box plot showing logarithms of concentrations of TNF-α in blood samples in an incident group (last eGFR is less than 60) and a stable group (last eGFR is 60 or more).

FIG. 4 is a box plot showing logarithms of concentrations of IL-22 in blood samples in an incident group (last eGFR is less than 60) and a stable group (last eGFR is 60 or more).

FIG. 5 is a box plot showing predicted probabilities in an incident group (last eGFR is less than 60) and a stable group (last eGFR is 60 or more).

### DESCRIPTION OF THE EMBODIMENTS

In a method for obtaining information on a risk of a decline in renal function in a CKD patient (hereinafter, also referred to as “the information obtaining method of the present embodiment”), a biomarker in a blood sample obtained from a CKD patient is measured. The blood sample is blood (whole blood), plasma, or serum obtained from a CKD patient. In the information obtaining method of the present embodiment, a measurement result of the biomarker in the blood sample serves as an index of the risk of the decline in renal function.

In the present specification, the term “patient with chronic kidney disease” includes both a patient diagnosed with CKD and a person suspected of having CKD. Preferably, the CKD patient is a patient diagnosed with CKD. CKD includes diabetic nephropathy. Diabetic nephropathy is a type of CKD and is known to account for about 33% of all CKD patients. CKD is diagnosed by presence of renal impairment and/or a value of eGFR (or GFR). Presence of renal impairment is confirmed by urinalysis, blood test, imaging diagnosis, pathological examination, or the like.

The severity of CKD in a patient at the time of obtaining a blood sample is not particularly limited. The severity of CKD can be classified into mild, moderate, severe, very severe, and renal failure based on, for example, eGFR (mL/min/1.73 m²) and albuminuria (mg/g or mg/mmol). The severity of CKD in a patient at the time of obtaining a blood sample is preferably mild or moderate. When the eGFR is 60 mL/min/1.73 m² or more and the albuminuria is less than 30 mg/g (or less than 3 mg/mmol), the CKD is mild. When the eGFR is 60 mL/min/1.73 m² or more and the albuminuria is 30 to 299 mg/g (or 3 to 29 mg/mmol), the CKD is moderate. Further, when the eGFR is 45 to 59 mL/min/1.73 m² and the albuminuria is less than 30 mg/g (or less than 3 mg/mmol), the CKD is moderate. By applying the information obtaining method of the present embodiment to a patient with mild or moderate CKD, a patient at risk of further decline in renal function can be extracted. In the information obtaining method of the present embodiment, the CKD patient may be, for example, a person whose eGFR at the time of obtaining the blood sample is 60 mL/min/1.73 m² or more.

The CKD patient may be receiving treatment for CKD. Examples of the treatment for CKD include improvement of lifestyle habits, diet therapy, drug therapy, and the like. Examples of the drug therapy include administration of a therapeutic agent for CKD such as a renin-angiotensin system (RAS) inhibitor, a statin, a non-steroidal mineralocorticoid receptor antagonist, a sodium-glucose cotransporter 2 (SGLT2) inhibitor, a glucagon-like peptide-1 (GLP-1) receptor agonist, and finerenone.

The biomarker measured by the information obtaining method of the present embodiment comprises at least one selected from the group consisting of IL-22 and TNF-α. Preferably, the biomarker is IL-22 and/or TNF-α. IL-22 is a cytokine belonging to the IL-10 family. IL-22 is mainly secreted by innate lymphoid cells (ILCs), helper T cells (particularly Th17 cells and Th22 cells), and natural killer (NK) cells. IL-22 does not act directly on immune cells, but acts on epithelial cells to maintain barrier function, contributing to regulation of immune response and tissue repair. TNF-α is one of major inflammatory cytokines. TNF-α is mainly secreted by immune cells such as macrophages, T cells, and NK cells. TNF-α acts on inflammatory response, induction of cell death, and activation of immune cells. The amino acid sequences of IL-22 and TNF-α per se are known and are disclosed in known databases such as a database provided by NCBI (National Center for Biotechnology Information) (www.ncbi.nlm.nih.gov).

When the blood sample contains insoluble contaminants such as cells, the contaminants may be removed by a known means such as centrifugation or filtration. The blood sample may be diluted with an appropriate aqueous medium as necessary. Such an aqueous medium is not particularly limited as long as it does not interfere with the measurement of the biomarker. Examples thereof include water, physiological saline, and a buffer. The buffer is not particularly limited as long as it has a buffering action at a pH near neutrality (for example, a pH of 6 or more and 8 or less). Examples of such a buffer include Good's buffers such as HEPES, MES, and PIPES, phosphate buffered saline (PBS), and Tris buffered saline (TBS).

In the present specification, “measuring a biomarker” includes determining a value of an amount or a concentration of a biomarker, and obtaining information reflecting an amount or a concentration of a biomarker. The “information reflecting an amount or a concentration of a biomarker” means an index that changes according to the amount or concentration of the biomarker in the blood sample. The above index is preferably an index of an optical change that is visible or measurable by a machine. Examples of the index of an optical change include luminescence intensity, fluorescence intensity, absorbance, turbidity, and intensity of color development.

Information reflecting an amount or a concentration of a biomarker may be qualitative information, quantitative information, or semi-quantitative information. Qualitative information reflecting an amount or a concentration of a biomarker is information indicating presence or absence of a biomarker. Quantitative information reflecting an amount or a concentration of a biomarker is numerical information such as a numerical value obtained by a measuring instrument (hereinafter, also referred to as “raw data”) and a value calculated from the numerical value. Examples of the value calculated from the raw data include a value obtained by subtracting a value of a negative control sample or a background value from the raw data. Based on the quantitative information, a value of an amount or a concentration of a biomarker can be determined. Semi-quantitative information reflecting an amount or a concentration of a biomarker is information indicating an amount or a concentration of a biomarker in stages by words, numbers (indicating grades), colors, or the like. For example, terms such as “below detection limit,” “low,” “moderate,” and “high” may be used.

A measurement result of a biomarker includes a value obtained by measuring the biomarker, information, and a combination thereof. Preferably, the measurement result of the biomarker is quantitative information reflecting an amount or a concentration of the biomarker, and a value of the amount or concentration of the biomarker determined based on the quantitative information. Hereinafter, the quantitative information reflecting the amount or concentration of the biomarker and the value of the amount or concentration of the biomarker are also referred to as a “measurement value of the biomarker.” Preferred measurement values of the biomarker are a measurement value of IL-22 and/or a measurement value of TNF-α. The measurement value of the biomarker may be a value obtained from a measurement value of IL-22 and a measurement value of TNF-α. Examples of such a value include a value obtained by a known multivariate analysis using a measurement value of IL-22 and a measurement value of TNF-α. As the value obtained by the multivariate analysis, a predicted probability is preferable. The predicted probability can be obtained by multiple logistic regression analysis. The predicted probability (P) can be calculated, for example, by the following regression formula.

P=1/[1+exp{−(a₁x₁+a₂x₂+ . . . +aₙxₙ+b)}]

In the above regression formula, x₁ to xₙ are measurement values of each biomarker, a₁ to aₙ are regression coefficients of each biomarker, and b is a constant. The regression coefficients and the constant can be set, for example, as follows. First, as shown in Examples described later, biomarkers in blood samples obtained from CKD patients whose renal function declined after obtaining the blood samples (incident group) and CKD patients whose renal function was maintained even after obtaining the blood samples (stable group) are measured. From data of the measurement values of each biomarker, a logistic model for distinguishing between the incident group and the stable group is created. Thereby, the regression coefficients and the constant can be set. The multiple logistic model can be created using statistical analysis software such as R (freeware also called “R language”) or SPSS Statistics (IBM Corporation). It is preferable to collect data of the measurement values of each biomarker in the incident group and the stable group, and to create a multiple logistic model in advance from the data.

The method for measuring a biomarker is not particularly limited as long as information reflecting an amount or a concentration of the biomarker in a blood sample can be obtained. Such a method may be, for example, a method of capturing a biomarker using a substance capable of specifically binding to the biomarker. For example, when IL-22 is measured as a biomarker, a substance capable of specifically binding to IL-22 is used. When TNF-α is measured as a biomarker, a substance capable of specifically binding to TNF-α is used. The biomarker in the blood sample can be measured by detecting the biomarker captured by such a substance by a known method.

Examples of the substance capable of specifically binding to a biomarker include an antibody, an aptamer, and a receptor protein. Among them, an antibody is particularly preferable. For example, when IL-22 is measured as a biomarker, it is preferable to use an antibody against IL-22 (hereinafter, also referred to as “anti-IL-22 antibody”). When TNF-α is measured as a biomarker, it is preferable to use an antibody against TNF-α (hereinafter, also referred to as “anti-TNF-α antibody”). In the present specification, “antibody” includes not only an immunoglobulin form but also an antibody fragment. Examples of the antibody fragment include Fab, F(ab′)2, Fab′, Fv, Fd, a domain antibody (dAb), a single-chain antibody (scFv), and a diabody. The antibody may be either a monoclonal antibody or a polyclonal antibody. An antibody against a biomarker per se is known and generally available. For example, anti-IL-22 antibodies and anti-TNF-α antibodies are commercially available.

The method for measuring a biomarker using an antibody is not particularly limited and can be selected from known immunoassay methods. Examples of the immunoassay method include an enzyme-linked immunosorbent assay (ELISA), an immune complex transfer method (see U.S. Pat. No. 5,236,849), and Western blotting. Among them, the ELISA is preferable. The type of the ELISA may be any of a sandwich method, a competitive method, a direct method, an indirect method, and the like. A sandwich method is preferable.

As an example, a case of measuring a biomarker in a blood sample by a sandwich ELISA will be described. In the sandwich ELISA, an antibody for capturing a biomarker (hereinafter, also referred to as “capture antibody”) and an antibody for detecting the biomarker (hereinafter, also referred to as “detection antibody”) are used. When both the capture antibody and the detection antibody are monoclonal antibodies, it is preferable that their epitopes are different from each other. In the measurement, first, a complex containing a biomarker, a capture antibody, and a detection antibody is formed on a solid phase. The complex can be formed by mixing a blood sample, a capture antibody, and a detection antibody. The step of forming the complex is preferably performed in a solution containing the biomarker, the capture antibody, and the detection antibody. By bringing a solution containing the formed complex into contact with a solid phase capable of immobilizing the capture antibody, the above complex can be formed on the solid phase. Alternatively, a solid phase on which a capture antibody is immobilized in advance may be used. In this case, by bringing the solid phase on which the capture antibody is immobilized, the blood sample, and the detection antibody into contact with each other, the above complex can be formed on the solid phase.

The solid phase may be any insoluble carrier capable of immobilizing a capture antibody. The mode of immobilization of the capture antibody to the solid phase is not particularly limited. For example, the capture antibody and the solid phase may be directly bonded, or the capture antibody and the solid phase may be indirectly bonded via another substance. Examples of the direct bonding include physical adsorption and the like. Examples of the indirect bonding include bonding via a combination of biotins and avidins. By modifying the capture antibody with biotins in advance and bonding avidins to the solid phase in advance, the capture antibody and the solid phase can be indirectly bonded via the bonding between the biotins and the avidins.

In the present specification, “biotins” include biotin and analogs thereof. Examples of the biotin analog include desthiobiotin and biocytin. In the present specification, “avidins” include avidin and analogs thereof. Examples of the avidin analog include streptavidin, an avidin-like protein derived from Pleurotus cornucopiae (Tamavidin (registered trademark)), bradavidin, and rhizavidin.

The material of the solid phase is not particularly limited, and can be selected from, for example, organic polymer compounds, inorganic compounds, biopolymers, and the like. Examples of the organic polymer compound include latex, polystyrene, and polypropylene. Examples of the inorganic compound include magnetic substances (iron oxide, chromium oxide, ferrite, and the like), silica, alumina, and glass. Examples of the biopolymer include insoluble agarose, insoluble dextran, gelatin, and cellulose. Two or more of these may be used in combination. The shape of the solid phase is not particularly limited, and examples thereof include a particle, a membrane, a microplate, a microtube, and a test tube. Among them, particles are preferable, and magnetic particles are particularly preferable.

Between the complex formation step and the complex detection step described later, B/F (Bound/Free) separation for removing unreacted free components not forming a complex may be performed. The unreacted free component refers to a component that does not constitute a complex. Examples thereof include a capture antibody and a detection antibody that have not bound to a biomarker. The means for B/F separation is not particularly limited. When the solid phase is a particle, only the solid phase capturing the complex is recovered by centrifugation. Thereby, B/F separation can be performed. When the solid phase is a container such as a microplate or a microtube, B/F separation can be performed by removing a liquid containing unreacted free components. When the solid phase is a magnetic particle, a liquid containing unreacted free components is suction-removed by a nozzle in a state where the magnetic particle is magnetically constrained by a magnet. Thereby, B/F separation can be performed. B/F separation by magnetic particles and a magnet is preferable from the viewpoint of automation. After removing the unreacted free components, the solid phase capturing the complex may be washed with an appropriate aqueous medium such as PBS.

The biomarker in the blood sample can be measured by detecting the complex formed on the solid phase by a known method. Detection of the complex is preferably performed in a solution. When an antibody labeled with a labeling substance is used as the detection antibody, a signal from the labeling substance is generated from the detection antibody contained in the complex on the solid phase. By detecting the signal, the complex on the solid phase is detected. Since this complex contains the biomarker captured from the blood sample by the capture antibody, the biomarker in the blood sample is measured by detecting the complex. When a labeled secondary antibody against the detection antibody is used, the biomarker in the blood sample can be measured in the same manner.

The labeling substance is not particularly limited as long as a detectable signal is generated. For example, it may be a substance that generates a signal itself (hereinafter, also referred to as “signal generating substance”), or a substance that generates a signal by catalyzing a reaction of another substance. Examples of the signal generating substance include a fluorescent substance and a radioactive isotope. Examples of the substance that catalyzes a reaction of another substance to generate a detectable signal include an enzyme. Examples of the enzyme include alkaline phosphatase, peroxidase, β-galactosidase, and luciferase. Examples of the fluorescent substance include fluorescent dyes such as fluorescein isothiocyanate (FITC), rhodamine, and Alexa Fluor (registered trademark), and fluorescent proteins such as GFP. Examples of the radioactive isotope include ¹²⁵I, ¹⁴C, and ³²P. Among them, an enzyme is preferable as the labeling substance, and alkaline phosphatase and peroxidase are particularly preferable.

The method for detecting a signal itself is known in the technical field. A measurement method according to the type of signal derived from the above labeling substance can be appropriately selected. For example, when the labeling substance is an enzyme, a signal such as light or color generated by reacting with a substrate for the enzyme is measured. For the signal measurement, a known instrument such as a spectrophotometer or a microplate reader can be used.

The substrate of the enzyme can be appropriately selected from known substrates according to the type of the enzyme. When the enzyme is alkaline phosphatase, for example, the following substances are used as the substrate.
CDP-Star (registered trademark) (4-chloro-3-(methoxyspiro[1,2-dioxetane-3,2′-(5′-chloro)trixyl[3.3.1.13,7]decan]-4-yl)phenyl phosphate disodium salt)
CSPD(registered trademark) (3-(4-methoxyspiro[1,2-dioxetane-3,2′-(5′-chloro)tricyclo[3.3.1.13,7]decan]-4-yl)phenylphosphate disodium salt)
5-bromo-4-chloro-3-indolyl phosphate (BCIP)
5-bromo-6-chloro-indolyl phosphate
p-nitrophenyl phosphate

When the enzyme is peroxidase, for example, the following substances are used as the substrate.
luminol and derivatives thereof
2,2′-Azinobis(3-ethylbenzothiazoline-6-sulfonic acid ammonium salt) (ABTS)
1,2-phenylenediamine (OPD)
3,3′,5,5′-tetramethylbenzidine (TMB)

When the labeling substance is a radioactive isotope, radiation as a signal can be measured using a known instrument such as a scintillation counter. When the labeling substance is a fluorescent substance, fluorescence as a signal can be measured using a known instrument such as a microplate reader. The excitation wavelength and the fluorescence wavelength can be appropriately determined according to the type of the fluorescent substance.

A detection result of a signal can be used as a measurement result of a biomarker. For example, when quantifying the intensity of a signal, a measurement value of signal intensity itself or a value obtained from the measurement value can be used as the measurement result of the biomarker. Examples of the value obtained from the measurement value of signal intensity include a value obtained by subtracting a measurement value of a negative control sample or a background value from the measurement value of signal intensity. The value of the amount or concentration of the biomarker may be determined by applying the measurement value of signal intensity to a calibration curve. The negative control sample can be appropriately selected. For example, a blood sample obtained from a healthy subject or the like can be mentioned.

A biomarker contained in a blood sample can be measured by a sandwich ELISA using a capture antibody immobilized on magnetic particles and a detection antibody labeled with a labeling substance. In this case, the measurement may be performed using a commercially available fully automated immunoassay apparatus. Examples of such a fully automated immunoassay apparatus include the HISCL (registered trademark) series from Sysmex Corporation.

As described above, the measurement result of the biomarker serves as an index of the risk of the decline in renal function in a CKD patient. Here, the risk of the decline in renal function is a risk that the eGFR of a CKD patient becomes less than 60 mL/min/1.73 m². More specifically, the risk of the decline in renal function is a risk that the eGFR of the patient becomes less than 60 mL/min/1.73 m² after a lapse of a predetermined period from the day when a blood sample was obtained from the CKD patient. The predetermined period is not particularly limited, and may be, for example, 977 days or more and 4123 days or less. Preferably, the predetermined period may be 2341 days or more and 3527 days or less. For example, as shown in Examples described later, the concentration of the biomarker in the blood sample was statistically significantly higher in the incident group than in the stable group. Therefore, the measurement result of the biomarker can be obtained as information on the risk of the decline in renal function in a CKD patient.

For example, when a measurement value of a biomarker is equal to or higher than a threshold value corresponding to the biomarker, the measurement value of the biomarker may suggest that the risk of the decline in renal function is high. When the measurement value of the biomarker is less than the threshold value corresponding to the biomarker, the measurement value of the biomarker may suggest that the risk of the decline in renal function is low. Alternatively, when the measurement value of the biomarker is higher than the threshold value corresponding to the biomarker, the measurement value of the biomarker may suggest that the risk of the decline in renal function is high. When the measurement value of the biomarker is equal to or less than the threshold value corresponding to the biomarker, the measurement value of the biomarker may suggest that the risk of the decline in renal function is low.

The threshold value corresponding to a biomarker is not particularly limited and can be set appropriately. For example, the threshold value may be set as follows. First, blood samples are collected from an incident group and a stable group, and a biomarker is measured to obtain measurement values of the biomarker. Then, a value capable of distinguishing between the incident group and the stable group with the highest accuracy is determined, and the value is set as the threshold value. In setting the threshold value, it is preferable to consider sensitivity, specificity, positive predictive value, negative predictive value, and the like.

Another embodiment of the present invention relates to a method for assisting determination of a risk of a decline in renal function in a CKD patient (hereinafter, also referred to as “determination assisting method”). This determination assisting method includes a step of measuring a biomarker in a blood sample obtained from a CKD patient, and a step of determining a risk of a decline in renal function in the CKD patient based on a measurement result. The step of measuring the biomarker in the blood sample is as described above. In the determination step, preferably, a measurement value of the biomarker is compared with a threshold value, and determination is made based on a comparison result. For example, when the measurement value of the biomarker is equal to or higher than a threshold value corresponding to the biomarker, it can be determined that the risk of the decline in renal function in the CKD patient is high. When the measurement value of the biomarker is less than the threshold value corresponding to the biomarker, it can be determined that the risk of the decline in renal function in the CKD patient is low. Alternatively, when the measurement value of the biomarker is higher than the threshold value corresponding to the biomarker, it can be determined that the risk of the decline in renal function in the CKD patient is high. When the measurement value of the biomarker is equal to or less than the threshold value corresponding to the biomarker, it can be determined that the risk of the decline in renal function in the CKD patient is low. Details of the CKD patient, the blood sample, the measurement of the biomarker, and the threshold value are as described above.

The present invention includes a method for treating CKD. The method for treating CKD of the present embodiment includes a step of performing treatment of CKD for a CKD patient suggested or determined to have a high risk of a decline in renal function. Examples of the treatment of CKD include improvement of lifestyle habits, diet therapy, drug therapy, and renal transplantation. These treatments may be employed alone or in combination. Examples of the drug therapy include administration of a therapeutic agent for CKD such as a RAS inhibitor, a statin, a non-steroidal mineralocorticoid receptor antagonist, a SGLT2 inhibitor, a GLP-1 receptor agonist, and finerenone. These therapeutic agents may be administered alone or in combination.

The present invention also includes a reagent kit for use in a method for obtaining information on a risk of a decline in renal function in a CKD patient or a method for assisting determination of a risk of a decline in renal function in a CKD patient (hereinafter, also referred to as “the reagent kit of the present embodiment”). The reagent kit of the present embodiment comprises, as a substance capable of specifically binding to a biomarker, a substance capable of specifically binding to IL-22 and/or a substance capable of specifically binding to TNF-α. Details of the substance capable of specifically binding to each biomarker are as described above.

A reagent containing a substance capable of specifically binding to IL-22 is preferably a reagent containing an anti-IL-22 antibody. More preferably, it is a combination of a reagent containing an anti-IL-22 antibody as a capture antibody and a reagent containing an anti-IL-22 antibody as a detection antibody. A reagent containing a substance capable of specifically binding to TNF-α is preferably a reagent containing an anti-TNF-α antibody. More preferably, it is a combination of a reagent containing an anti-TNF-α antibody as a capture antibody and a reagent containing an anti-TNF-α antibody as a detection antibody. When both the capture antibody and the detection antibody are monoclonal antibodies, it is preferable that their epitopes are different from each other.

Preferably, the reagent kit of the present embodiment comprises a reagent containing a capture antibody and a reagent containing a detection antibody. The detection antibody may be labeled with a labeling substance. When the labeling substance is an enzyme, the reagent kit of the present embodiment may contain a substrate for the enzyme. Details of the capture antibody, the detection antibody, the labeling substance, and the substrate are as described above. The forms of the capture antibody, the detection antibody, the labeling substance, and the substrate are not particularly limited. Their forms can be, for example, a solid (for example, powder, crystal, lyophilizate, etc.) or a liquid (for example, solution, suspension, emulsion, etc.).

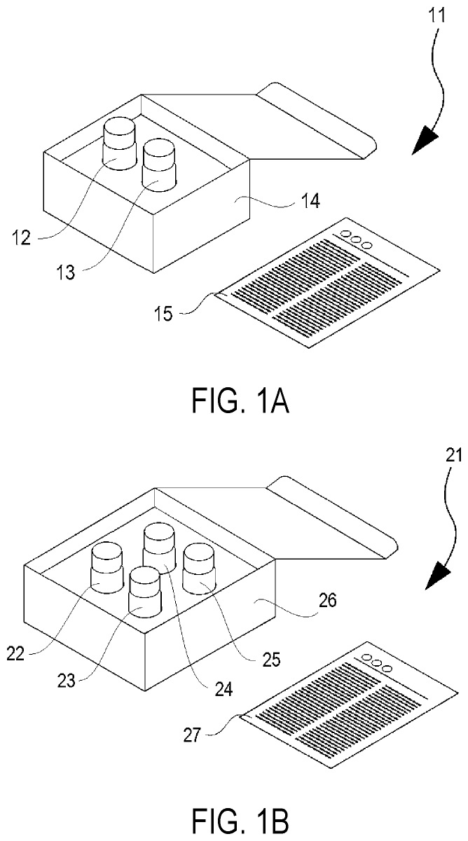

The reagent kit of the present embodiment can be provided to a user in a state where a container containing the reagent is packed in a box. A package insert may be enclosed in the box. The package insert may describe the configuration of the reagent kit, the method of use, the relationship between measurement results obtained by the reagent kit and the pathological condition of a patient, and the like. An example of such a reagent kit is shown in FIG. 1A. The reagent kit shown in FIG. 1A includes a reagent containing a capture antibody against IL-22 and a reagent containing a detection antibody against IL-22, but the present invention is not limited to this example. The reagent kit may include a reagent containing a capture antibody against TNF-α and a reagent containing a detection antibody against TNF-α. Referring to FIG. 1A, 11 denotes a reagent kit, 12 denotes a first container containing a reagent containing a capture antibody, 13 denotes a second container containing a reagent containing a detection antibody, 14 denotes a packaging box, and 15 denotes a package insert. In this example, the reagent kit may further include a solid phase for immobilizing the capture antibody. Details of the solid phase are as described above.

The reagent kit shown in FIG. 1B includes a reagent containing a capture antibody against IL-22, a reagent containing a detection antibody against IL-22, a reagent containing a capture antibody against TNF-α, and a reagent containing a detection antibody against TNF-α. Referring to FIG. 1B, 21 denotes a reagent kit, 22 denotes a first container containing a reagent containing a capture antibody against IL-22, 23 denotes a second container containing a reagent containing a detection antibody against IL-22, 24 denotes a third container containing a reagent containing a capture antibody against TNF-α, 25 denotes a fourth container containing a reagent containing a detection antibody against TNF-α, 26 denotes a packaging box, and 27 denotes a package insert. In this example, the reagent kit may further include a solid phase for immobilizing the capture antibody.

The reagent kit may further include a calibrator. The calibrator is a reagent for quantifying a biomarker and contains a standard substance for the biomarker. For example, when the reagent kit includes a reagent containing a substance capable of specifically binding to IL-22, the calibrator is a buffer solution containing a recombinant protein or synthetic protein of IL-22 at a predetermined concentration as a standard substance. When the reagent kit includes a reagent containing a substance capable of specifically binding to TNF-α, the calibrator is a buffer solution containing a recombinant protein or synthetic protein of TNF-α at a predetermined concentration as a standard substance. There may be one calibrator or a plurality of calibrators. When the reagent kit includes a plurality of calibrators, it is preferable that the concentrations of the standard substance are different from each other in each calibrator. The calibrator may further contain a buffer solution (negative control) that does not contain a standard substance.

The present invention includes the use of an anti-IL-22 antibody and/or an anti-TNF-α antibody for producing the reagent kit described above. Details of the reagent kit are as described above.

Hereinafter, the present invention will be described in detail by way of examples, but the present invention is not limited to these examples.

### Example 1(1) Blood Sample

As blood samples, specimens of diabetic nephropathy patients (66 patients) in the B. Fernandez et al. study were used. These specimens were serum prepared by a conventional method from blood collected from each patient at baseline. “At baseline” was at the start of eGFR monitoring or after a lapse of a predetermined period from the start of eGFR monitoring. The baseline was set for each patient. About half of the patients had blood collected at the start of eGFR monitoring. The median period from the start of eGFR monitoring to blood collection was 135 days. The blood samples were obtained from Fundacion Jimenez Diaz Biobank (Tax number NIF: U83633859, Address: Fundacion Jimenez Diaz-UTE, Avenida de Reyes Catolicos 2, Madrid, Spain, Representative: Federico Rojo). The B. Fernandez et al. study was a single-center prospective observational study. The research purpose was to understand progression factors of diabetic nephropathy, particularly to understand whether there are gender differences, in order to develop effective prevention and treatment strategies (B. Fernandez-Fernandez et al., J. Clin. Med. 2020, 9(6), 1611).

### (2) Patient Information

The above patients were adult patients diagnosed with type I or type II diabetes and diagnosed with CKD. These patients included those diagnosed with albuminuria (urine albumin-to-creatinine ratio (UACR)>30 mg/g). In each patient, there were no other causes indicating CKD. The eGFR of each patient was calculated by the CKD EPI method based on blood creatinine concentration (mg/dL), age, sex, and Black (Yes/No). The UACR of each patient was calculated by dividing the albumin level (mg/L) in urine collected at a predetermined time by the creatinine level (g/L). Blood creatinine and urinary creatinine were measured by quantitative methods such as colorimetry. Each patient was followed up until death or start of renal transplantation.

### (3) Measurement of Biomarkers

IL-22, TNF-α, IL-6, IL-17, TWEAK, and GDF15 in blood samples of each patient were measured. IL-6, IL-17, TWEAK, and GDF15 were biomarkers reported to be associated with diabetic nephropathy. Measurement of IL-22, IL-17, and TWEAK was performed by a fully automated immunoassay apparatus HISCL-5000 (Sysmex Corporation). TNF-α and IL-6 were measured using ELISA kits purchased from Bio-Techne Corporation. GDF15 was measured using ELISA reagents purchased from R&D Systems, Inc.

As reagents for measuring IL-22, IL-17, and TWEAK, R1 reagents, R2 reagents, and R3 reagents in (3.1) to (3.3) below were prepared. The R1 reagent was a capture reagent containing an antibody specifically binding to each biomarker. The R2 reagent was a reagent containing magnetic particles as a solid phase. The R3 reagent was a detection reagent containing an ALP-labeled antibody specifically binding to each biomarker. In addition, for measurement of IL-22, IL-17, and TWEAK, HISCL R4 reagent (Sysmex Corporation), which is a buffer for measurement, and HISCL R5 reagent (Sysmex Corporation) containing CDP-Star (registered trademark) (Applied Biosystems, Inc.), which is a chemiluminescent substrate for ALP, were used.

### (3.1) Reagent for IL-22 Measurement

### R1 Reagent

An anti-IL-22 monoclonal antibody (Clone: 142906, Catalog #MA5-23775, Thermo Fisher Scientific Inc.) was labeled with biotin by a conventional method. The obtained biotin-labeled anti-IL-22 antibody was dissolved in a buffer to prepare an R1 reagent (antibody concentration 6 μg/ml). As the buffer, HISCL R1 buffer containing 50 mM EGTA and 0.1% Tween20 was used.

### R2 Reagent

Magnetic particles having streptavidin immobilized on the surface (hereinafter, also referred to as “STA-bound magnetic particles”; average particle size 2 m; amount of streptavidin per 1 g of magnetic particles is 2.9 to 3.5 mg) were washed three times with 10 mM HEPES (pH 7.5). The STA-bound magnetic particles were suspended in 10 mM HEPES (pH 7.5) such that the concentration of the STA-bound magnetic particles was 1 mg/ml, to prepare an R2 reagent.

### R3 Reagent

An anti-IL-22 monoclonal antibody (Clone: 24, Catalog #MA5-30814, Thermo Fisher Scientific Inc.) was labeled with ALP by a conventional method. The obtained ALP-labeled anti-IL-22 antibody was dissolved in a buffer to prepare an R3 reagent (antibody concentration 1 pmol/ml). As the buffer, HISCL R3 buffer containing 50 mM EGTA and 0.1% Tween20 was used.

### (3.2) Reagent for IL-17 Measurement

### R1 Reagent

A biotin-labeled anti-IL-17 monoclonal antibody (Clone: eBio64DEC17, Catalog #13-7179-81, Invitrogen) was dissolved in 100 mM triethanolamine (pH 7.4) (containing 150 mM NaCl, 1% BSA, and 0.5% casein) to prepare an R1 reagent.

### R2 Reagent

An R2 reagent for IL-17 measurement was prepared in the same manner as the R2 reagent for IL-22 measurement, except that the concentration of the STA-bound magnetic particles was 0.5 mg/ml.

### R3 Reagent

An ALP-labeled anti-IL-17 monoclonal antibody (Clone: eBio64CAP17, Catalog #14-7178-81, Invitrogen Corporation) was dissolved in 100 mM triethanolamine (pH 7.4) (containing 150 mM NaCl and 1% BSA) to prepare an R3 reagent.

(3.3) Reagent for TWEAK measurement

### R1 Reagent

An anti-human TWEAK monoclonal antibody (Clone: 173830, Catalog #MAB1090-500, R&D Systems, Inc.) was labeled with biotin by a conventional method. The obtained biotin-labeled anti-TWEAK antibody was dissolved in 100 mM triethanolamine (pH 7.4) (containing 150 mM NaCl, 1% casein, 10 mM EDTA, and 10 mM EGTA) to prepare an R1 reagent.

### R2 Reagent

An R2 reagent for TWEAK measurement was prepared in the same manner as the R2 reagent for IL-22 measurement, except that the concentration of STA-bound magnetic particles was 0.5 mg/ml.

### R3 Reagent

An anti-TWEAK monoclonal antibody (Clone: EPR22899-249, Catalog #ab281090, Abcam) was labeled with ALP by a conventional method. The obtained ALP-labeled anti-TWEAK antibody was dissolved in 100 mM triethanolamine (pH 7.4) (containing 150 mM NaCl and 1% BSA) to prepare an R3 reagent.

### (3.4) Measurement Procedure by HISCL-5000

After a blood sample and the R1 reagent were mixed, the R2 reagent was added thereto. Magnetic particles in the obtained mixture were collected by magnetism, a supernatant was removed, and an HISCL washing solution was added to wash the magnetic particles. The supernatant was removed, and the R3 reagent was added to and mixed with the magnetic particles. Magnetic particles in the obtained mixture were collected by magnetism, the supernatant was removed, and the HISCL washing solution was added to wash the magnetic particles. The supernatant was removed, the R4 reagent and the R5 reagent were added to the magnetic particles, and chemiluminescence intensity was measured. The obtained chemiluminescence intensity was applied to a calibration curve to determine a concentration of a biomarker. The calibration curve was created using a calibrator for each biomarker. The calibrator was prepared from a recombinant protein of each biomarker.

### (4) Classification of Patients Based on Disease Progression

For each patient, eGFR at baseline (referred to as “initial eGFR”) and eGFR at 977 days or more after the start of the study by B. Fernandez et al. (referred to as “final eGFR”) were obtained. Each patient was classified into an “incident group” or a “stable group” based on the initial eGFR and the final eGFR. The incident group was a patient group in which the initial eGFR was 60 mL/min/1.73 m² or more and the final eGFR was less than 60 mL/min/1.73 m². The stable group was a patient group in which the initial eGFR was 60 mL/min/1.73 m² or more and the final eGFR was also 60 mL/min/1.73 m² or more.

### (5) Measurement Results of Biomarkers

Table 1 shows measurement results (median, first quartile (1Q), third quartile (3Q), and number of patients) of each biomarker in the disease progression group (Incident group) and the non-disease progression group (Stable group). For each biomarker, a statistical significant difference between the incident group and the stable group was evaluated by the Mann-Whitney U test, and a p-value was calculated. As shown in Table 1, among the six biomarkers, the p-value was 0.05 or less for IL-22 and TNF-α. Therefore, it was shown that there is a statistical significant difference in the measurement values of IL-22 and TNF-α between the incident group and the stable group. In the following, it was examined whether a risk of a decline in eGFR can be determined based on the measurement values of IL-22 and TNF-α.

(6) Determination of a Risk of a Decline in eGFR

TABLE 1

| | Incident group | | | Stable group | | | |
|---|---|---|---|---|---|---|---|
| Biomarker | Median | (1Q-3Q) | Patients | Median | (1Q-3Q) | Patients | p-value |
| IL-6 | 5.3 | (2.9-5.83) | 13 | 3.19 | (1.71-6.66) | 26 | 0.776 |
| IL-17 | 0.46 | (0.39-1.97) | 13 | 0.59 | (0.36-1.15) | 25 | 0.976 |
| IL-22 | 23.02 | (19.05-26.46) | 13 | 16.13 | (11.21-18.34) | 26 | 0.044 |
| TNF-α | 17.65 | (15.53-19.83) | 13 | 14.07 | (11.84-16.47) | 26 | 0.032 |
| TWEAK | 272.74 | (247.37-311.21) | 13 | 227.79 | (230.27-340.69) | 25 | 0.957 |
| GDF15 | 2019.02 | (1491.1-3335.73) | 13 | 1880.25 | (1154.4-2359.42) | 26 | 0.792 |

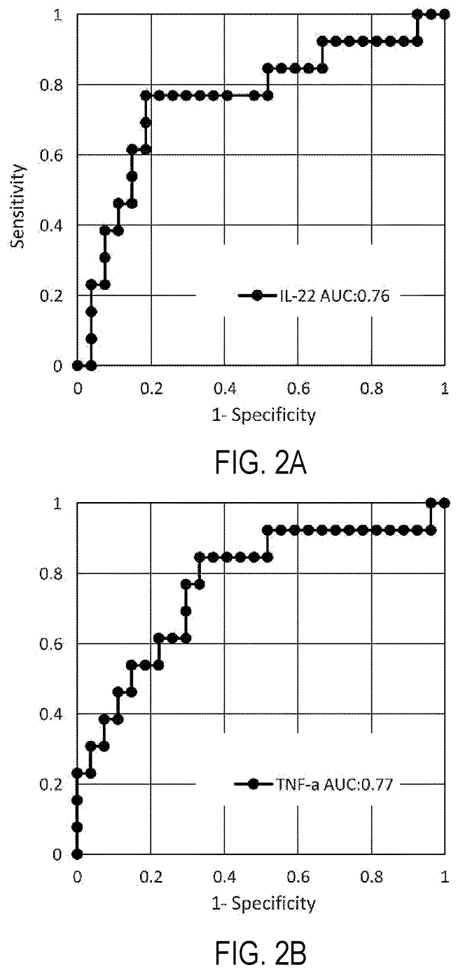

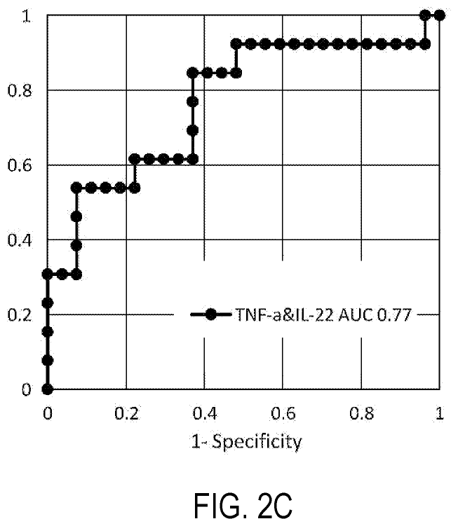

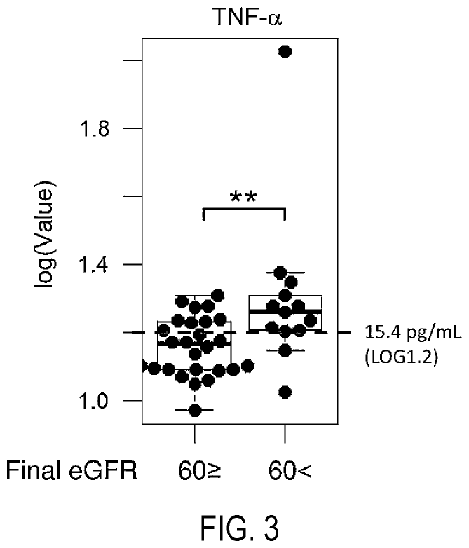

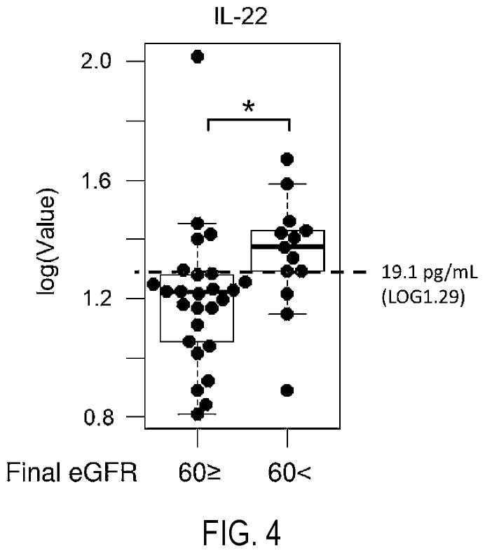

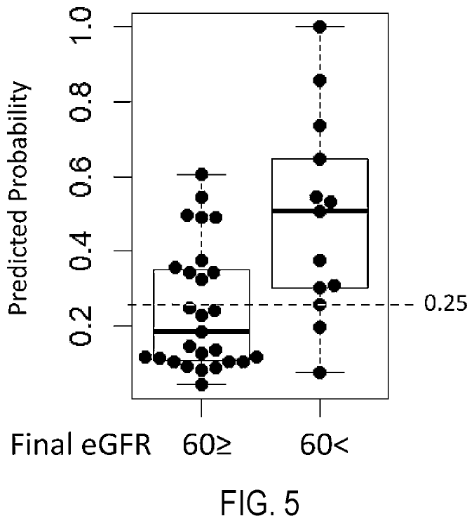

By ROC analysis, cutoff values for determining a risk of a decline in eGFR were determined based on a TNF-α concentration, an IL-22 concentration, and a combination thereof. Obtained ROC curves are shown in FIGS. 2A to 2C. Results of the ROC analysis were evaluated by a value of an area under the curve (AUC). As shown in FIG. 2A, the AUC of the determination based on the TNF-α concentration was 0.76. The cutoff value for TNF-α was determined to be 15.4 pg/mL. As shown in FIG. 2B, the AUC of the determination based on the IL-22 concentration was 0.77. The cutoff value for IL-22 was determined to be 19.1 pg/mL. Multiple logistic regression analysis was performed using measurement values of TNF-α and IL-22 as explanatory variables to obtain a predicted probability. Statistical analysis software R4.0.3 was used for this analysis. As shown in FIG. 2C, the AUC of the determination based on the predicted probability was 0.77. The cutoff value for the predicted probability was determined to be 0.25. Logarithms of the concentrations of TNF-α and IL-22 in the blood samples in the incident group and the stable group are shown in FIGS. 3 and 4. “**” in FIG. 3 indicates P<0.01, and “*” in FIG. 4 indicates P<0.05. Predicted probabilities in the incident group and the stable group are shown in FIG. 5. Dashed lines in FIGS. 3 to 5 indicate the cutoff values.

There were 20 patients whose TNF-α concentration at baseline was equal to or higher than the cutoff value. Of these, the final eGFR of 11 patients became less than 60 mL/min/1.73 m² after 977 days or more from the start of the study. That is, the renal function declined. There were 20 patients whose TNF-α concentration at baseline was lower than the cutoff value. Of these, the final eGFR of 18 patients remained 60 mL/min/1.73 m² or more even after 977 days or more from the start of the study. That is, the renal function did not deteriorate. Sensitivity and specificity were calculated for the case where the risk of the decline in eGFR was determined based on the TNF-α concentration. Results are shown in Table 2.

TABLE 2

| Final eGFR | 60≥ | 60< | |
|---|---|---|---|
| Cut off< | 18 | 2 | 20 |
| Cut off≥ | 9 | 11 | 20 |
| | 27 | 13 | |
| Sensitivity: | 85% | | |
| Specificity: | 67% | | |

There were 15 patients whose IL-22 concentration at baseline was equal to or higher than the cutoff value. Of these, the final eGFR of 10 patients became less than 60 mL/min/1.73 m² after 977 days or more from the start of the study. That is, the renal function declined. There were 25 patients whose IL-22 concentration at baseline was lower than the cutoff value. Of these, the final eGFR of 22 patients remained 60 mL/min/1.73 m² or more even after 977 days or more from the start of the study. That is, the renal function did not deteriorate. Sensitivity and specificity were calculated for the case where the risk of the decline in eGFR was determined based on the IL-22 concentration. Results are shown in Table 3.

TABLE 3

| FinaleGFR | 60≥ | 60< | |
|---|---|---|---|
| Cut off< | 22 | 3 | 25 |
| Cut off≥ | 5 | 10 | 15 |
| | 27 | 13 | |
| Sensitivity: | 77% | | |
| Specificity: | 81% | | |

There were 21 patients whose predicted probability at baseline was equal to or higher than the cutoff value. Of these, the final eGFR of 11 patients became less than 60 mL/min/1.73 m² after 977 days or more from the start of the study. That is, the renal function declined. There were 19 patients whose predicted probability at baseline was lower than the cutoff value. Of these, the final eGFR of 17 patients remained 60 mL/min/1.73 m² or more even after 977 days or more from the start of the study. That is, the renal function did not deteriorate. Sensitivity and specificity were calculated for the case where the risk of the decline in eGFR was determined based on the predicted probability. Results are shown in Table 4.

TABLE 4

| Final eGFR | 60≥ | 60< | |
|---|---|---|---|
| Cut off< | 17 | 2 | 19 |
| Cut off≥ | 10 | 11 | 21 |
| | 27 | 13 | |
| Sensitivity: | 85% | | |
| Specificity: | 63% | | |

As shown in FIGS. 3 to 5 and Tables 2 to 4, it was shown that CKD patients can be classified into a group with a high risk of a decline in renal function and a group with a low risk of the decline by performing determination based on the measurement results of TNF-α in blood samples, the measurement results of IL-22, and a combination thereof.

What is claimed is:

1. A method for obtaining information on a risk of a decline in renal function in a patient with chronic kidney disease, comprising a step measuring a biomarker in a blood sample obtained from the patient with chronic kidney disease, wherein the biomarker comprises at least one selected from the group consisting of IL-22 and TNF-α, and a measurement result of the biomarker serves as an index for the risk of the decline in renal function.

2. The method according to claim 1, wherein the measuring step is a step of immunologically measuring the biomarker.

3. The method according to claim 1, wherein when a measurement value of the biomarker is equal to or higher than a threshold value corresponding to the biomarker, it is suggested that the risk of the decline in renal function is high.

4. The method according to claim 1, wherein when a measurement value of the biomarker is lower than a threshold value corresponding to the biomarker, it is suggested that the risk of the decline in renal function is low.

5. The method according to claim 1, wherein the patient with chronic kidney disease is a subject whose eGFR at a time of obtaining the blood sample is 60 mL/min/1.73 m² or more.

6. The method according to claim 1, wherein the risk of the decline in renal function is a risk that eGFR becomes less than 60 mL/min/1.73 m².

7. The method according to claim 3, further comprising a step of performing a treatment of chronic kidney disease for the patient suggested that the risk of the decline in renal function is high.

8. The method according to claim 7, wherein the treatment comprises improvement of lifestyle habits, diet therapy, drug therapy, and renal transplantation.

9. The method according to claim 8, wherein the drug therapy comprises administration of at least one therapeutic agent selected from the group consisting of a RAS inhibitor, a statin, a non-steroidal mineralocorticoid receptor antagonist, a SGLT2 inhibitor, a GLP-1 receptor agonist, and finerenone.

10. A method for assisting determination of a risk of a decline in renal function in a patient with chronic kidney disease, comprising the steps of:
measuring a biomarker in a blood sample obtained from the patient; and
determining the risk of the decline in renal function in the patient based on a measurement result,
wherein the biomarker comprises at least one selected from the group consisting of IL-22 and TNF-α.

11. The method according to claim 10, wherein the measuring step is a step of immunologically measuring the biomarker.

12. The method according to claim 10, wherein in the determining step, when a measurement value of the biomarker is equal to or higher than a threshold value corresponding to the biomarker, it is determined that the risk of the decline in renal function is high.

13. The method according to claim 10, wherein in the determining step, when a measurement value of the biomarker is lower than a threshold value corresponding to the biomarker, it is determined that the risk of the decline in renal function is low.

14. The method according to claim 10, wherein the patient with chronic kidney disease is a subject whose eGFR at a time of obtaining the blood sample is 60 mL/min/1.73 m² or more.

15. The method according to claim 7, wherein the risk of the decline in renal function is a risk that eGFR becomes less than 60 mL/min/1.73 m².

16. The method according to claim 12, further comprising a step of performing a treatment of chronic kidney disease for the patient determined that the risk of the decline in renal function is high.

17. The method according to claim 16, wherein the treatment comprises improvement of lifestyle habits, diet therapy, drug therapy, and renal transplantation.

18. The method according to claim 17, wherein the drug therapy comprises administration of at least one therapeutic agent selected from the group consisting of a RAS inhibitor, a statin, a non-steroidal mineralocorticoid receptor antagonist, a SGLT2 inhibitor, a GLP-1 receptor agonist, and finerenone.
